(19) United States
(12) Patent Application Publication
(10) Pub. No.: US 2009/0275895 A1
(43) Pub. Date: Nov. 5, 2009

(54) Drug Delivery Device for Infusion of Several Portions of Drug

(75) Inventors: Gunner Sie, Virum (DK); Louise Charlotte klok, Soborg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 12/298,334

(22) PCT Filed: Apr. 13, 2007
(86) PCT No.: PCT/EP2007/053627

§ 371 Date: Feb. 20, 2009

(30) Foreign Application Priority Data
Apr. 24, 2006 (EP) 06008399.5

(51) Int. Cl. A61M 5/142; A61M 5/00

(52) U.S. Cl. 604/151; 604/187

(57) ABSTRACT

A drug delivery device (1) for delivering fluid drug, preferably liquid drug. The device (1) comprises means for receiving and retaining a reservoir (5) containing drug. The retaining means (13, 14) is movable between a locking state, in which the reservoir (5) is retained, and an unlocking state, in which the reservoir (5) is released. Thereby drug from several reservoirs (5) may be delivered while the device (1) is operated using only one hand. Thereby the risk of displacing an infusion needle during change of reservoir is minimised. The device (1) comprises pumping means (3) for causing a flow of fluid in an established fluid connection. The drug delivery device (1) is preferably movable between an ‘aspiration mode’ and an ‘infusion mode’, most preferably automatically during insertion/release of a reservoir (5). This enables integrated (blood) aspiration, minimum waste of drug and minimum unintended infusion of air in the vein.

### FIELD OF THE INVENTION

The present invention relates to a device for delivering fluid drug, e.g. by means of infusion. More particularly, the present invention relates to a drug delivery device which is suitable for delivering several portions of drug consecutively. Furthermore, the present invention relates to a drug delivery device which is operable using only one hand.

### BACKGROUND OF THE INVENTION

When administering fluid drug to a person, e.g. liquid drug for infusion or injection, or gaseous drug for inhalation, it is sometimes desirable to deliver a dose of drug which exceeds the drug contained in a single reservoir, e.g. a vial or a canister. Infusion of a liquid drug is often performed using a needle, such as a butterfly needle, inserted into a body part of the person. At an opposite end the butterfly needle is connected to a reservoir containing the drug to be delivered, possibly via a drug delivery device. In case the desired dose is larger than the contents of a single reservoir, it is necessary to replace one, empty, reservoir by another, full, reservoir during the infusion. This will normally require two hands, and it is therefore not possible for the person to hold the butterfly needle during the replacement operation. This introduces the risk that the needle is accidentally moved during the replacement operation, e.g. causing the needle to be pulled out of a vein where it is supposed to be inserted, thereby risking that the next dose of drug is erroneously delivered in another kind of tissue, e.g. a subcutaneous layer instead of a vein. In some cases the needle may even be entirely dismantled from the person.

Furthermore, it is a challenge to eliminate air which is drawn out of the reservoir in order to make use of as large a part of the drug as possible, or at least to prevent such air from being injected along with the drug.

### SUMMARY OF THE INVENTION

It is, thus, an object of the invention to provide a drug delivery device which is suitable for delivery of a dose of drug which exceeds the amount of drug normally contained in a standard reservoir.

It is a further object of the invention to provide a drug delivery device which is operable using only one hand, even if several individual doses of drug must be delivered.

According to the invention the above and other objects are fulfilled by providing a drug delivery device comprising:
means for receiving a reservoir containing a drug to be delivered,
means for retaining a reservoir in the receiving means, said retaining means being adapted to be in a locking state in which a reservoir may be retained, and in an unlocking state in which the reservoir is released,
means for operating the retaining means to move between the locking state and the unlocking state, thereby causing a reservoir to be retained in or released from the receiving means,
an outlet opening adapted to deliver a drug from the device,
means for establishing a fluid connection between a reservoir retained in the receiving means and the outlet opening, thereby allowing a fluid flow between the reservoir and the outlet opening, and
pumping means for causing a flow of fluid in the established fluid connection.

The reservoir may be a vial, a canister, a flexible reservoir, such as a bag, or any other kind of reservoir which is suitable for holding the relevant drug.

The drug is preferably a fluid drug, such as a liquid drug for infusion or injection, or a gaseous drug for inhalation. Alternatively the drug may be a powdered or lyophilized drug which must be reconstituted, i.e. it must be mixed with a liquid, prior to infusion or injection of the drug. In this case the drug may be reconstituted before the reservoir is positioned in the receiving means. Alternatively, the drug may be reconstituted while the reservoir is retained in the receiving means. In any case, the drug delivered from the drug delivery device is in liquid form.

The receiving means is a part of the drug delivery device which is shaped in such a manner that a desired kind of reservoir will fit into the receiving means in a manner which allows the contents of the reservoir to be delivered by the drug delivery device via the outlet opening.

The drug delivery device comprises means for retaining a reservoir in the receiving means. The retaining means may be in a locking state and in an unlocking state. When the retaining means is in the locking state, a reservoir positioned in the receiving means will be retained by the retaining means. Thus, the reservoir will be securely fitted in the receiving means, and it is not necessary to manually hold it in position during delivery of the drug. On the other hand, when the retaining means is in the unlocking state the reservoir is released, i.e. it can be removed from the receiving means, and it may be replaced by another, full, reservoir.

The retaining means is operable, preferably using only one hand, to move between the locking state and the unlocking state. Thus, a user may easily position and remove reservoirs, even during the infusion, and without having to remove a needle inserted in a body part of the person, and without risking that a needle is accidentally displaced as described above. This is very advantageous.

The drug delivery device further comprises means for establishing a fluid connection between a reservoir retained in the receiving means and the outlet opening. Thereby fluid may flow between the reservoir and the outlet opening. It should be noted that the fluid may flow in either direction, i.e. from the reservoir towards the outlet opening or from the outlet opening towards the reservoir, depending on which function is desired. This will be described in further detail below.

Furthermore, the drug delivery device comprises pumping means for causing a flow of fluid in the established fluid connection. The pumping means may, e.g., be or comprise a piston pump or a peristaltic pump. The pumping means may be motor driven. This is advantageous in case the person using the pumping means has low dexterity, e.g. an elderly person, a child, or a person having reduced motoric skills and/or strength of the hands and/or fingers. It may be necessary to provide the reservoir with venting means or other means for adapting pneumatic pressure for the pumping means.

Thus, fluid is forced via the established fluid connection, either in a direction from the reservoir towards the outlet opening, or in an opposite direction from the outlet opening towards the reservoir.

According to one embodiment the pumping means may be adapted to be in a first state in which fluid is allowed to flow from a reservoir retained in the receiving means towards the outlet opening, and in a second state in which fluid is allowed to flow from the outlet opening towards the reservoir, the pumping means being switchable between the first state and the second state. According to this embodiment it is possible to switch the direction of the flow of fluid. When the pumping means is in the first state the drug which is contained in the reservoir is allowed to pass via the established fluid connection to the outlet opening. Thereby the drug is delivered from the drug delivery device. When the pumping means is in the second state fluid is sucked from the outlet opening towards the reservoir. Thereby aspiration may be performed. When a reservoir has been positioned in the receiving means it is necessary to aspirate, i.e. draw some blood or other suitable body fluid into the device, in order to ensure that a correct injection site has been selected, and in order to prevent air from being injected along with the drug. Furthermore, in case the reservoir is replaced by another, full, reservoir, it is advantageous to aspirate before delivering the drug contained in the new reservoir in order to prevent air from being injected along with the drug. When a reservoir is emptied in a manner which makes use of the available drug to the greatest extent possible, it is unavoidable that some air is drawn from the reservoir into the drug delivery device. In this case it is very advantageous to be able to aspirate some of the drug from the previous reservoir before inserting the next reservoir. Thereby it is possible to utilise the available drug to the greatest extent possible while avoiding that air is injected along with the drug.

Thus, according to this embodiment, the drug delivery device may be used for delivering a drug as well as for aspiration, and the drug delivery device is easily operable to be switched between the two states providing this. This is very advantageous, in particular when the relevant drug is relatively expensive.

In the present context the term ‘switchable between the first state and the second state’ should be interpreted broadly. Thus, it should be interpreted to include physically moving the pumping means or a part of the pumping means between two physically distinct positions. In this case the part which is physically moved may advantageously be a valve element being switched between two positions. This may, e.g., be obtained by means of rotation or linear displacement of the valve element, thereby causing the valve element to be switched between two positions, each allowing the flow of fluid in one of the directions mentioned above. On the other hand, it should also be interpreted to include switching between two states without physically moving one or more parts.

The pumping means may be automatically switched between the first state and the second state in response to positioning or removing a reservoir in/from the receiving means. Preferably, the pumping means is automatically switched to the first state when a reservoir is positioned in the receiving means, and switched to the second state when a reservoir is removed from the receiving means. Accordingly, when no reservoir is present in the receiving means, the device performs aspiration. When a reservoir is subsequently positioned in the receiving means, the pumping means is automatically switched to the first state, i.e. the drug in the reservoir can be delivered. When the reservoir is empty, it is released from the receiving means, and the pumping means is automatically moved back to the second state, the device thereby once again performing aspiration.

It is advantageous that the drug delivery device according to this embodiment is adapted to automatically switch between ‘infusion mode’ and ‘aspiration mode’, since it is thereby ensured that it is possible to switch between these to operations without additional necessary steps. Furthermore, switching between the two operations may be done using only one hand.

Alternatively or additionally, the pumping means may be manually operable. This may, e.g., be obtained by means of a manually operable valve switching element.

The retaining means may comprise releasable locking means, such as releasable latching means. Such mechanisms are easily activated and released, and they are therefore easily operable using only one hand. Alternatively, the retaining means may comprise a lock-track mechanism, e.g. similar to the locking mechanism found in some ballpoint pens.

The retaining means may comprise means for ejecting a reservoir being retained in the receiving means. In this case, when the retaining means is moved to the unlocking state, the reservoir is simultaneously ejected from the receiving means. This may, e.g., be obtained by means of a biasing mechanism, e.g. incorporating a spring. This is advantageous because it makes it even easier to operate the drug delivery device using only one hand.

The means for establishing a fluid connection may comprise means for penetrating a reservoir being retained in the receiving means. This may, e.g., be in the form of a hollow needle adapted to penetrate a wall of a flexible reservoir or a stopper of a vial, thereby establishing a fluid connection to the interior of the reservoir.

The penetrating means may advantageously comprise means for shielding the penetrating means when not penetrating a reservoir. This may, e.g., be in the form of a protecting flange positioned in the vicinity of the penetrating means, or the penetrating means may be retractable in such a manner that, e.g. a tip of a needle is not exposed. Thereby injuries due to cutting or sticking to persons using the drug delivery device can be avoided.

The outlet opening may be connectable to tubing for delivering the drug. In this case the tubing may advantageously be connected to a needle device, such as a butterfly needle, at an opposite end, the tubing thereby interconnecting the outlet opening and a needle inserted in a body part of a person to receive the drug. In this case the tubing must be connected to the drug delivery device before drug can be delivered. The tubing may be connected by means of a luer lock connection, by means of mating threads, or in any other suitable manner.

Alternatively, the outlet opening may be provided with needle means for delivering the drug. In this case a needle is directly positioned at the outlet opening, and the needle forms part of the drug delivery device. In this case the drug delivery device, including the needle, may be disposable.

The drug delivery device may be contained in a handle. In this case a very compact device which is easily operated using only one hand is obtained.

### BRIEF DESCRIPTION OF THE DRAWINGS

The invention will now be described in further details with reference to the accompanying drawing in which

FIG. 1 is a perspective view of a drug delivery device according to an embodiment of the invention,

FIGS. 2-4 are cross sectional views of the drug delivery device of FIG. 1, and

FIGS. 5-8 illustrate various valve arrangements for use in drug delivery devices according to the invention.

### DETAILED DESCRIPTION OF THE DRAWINGS

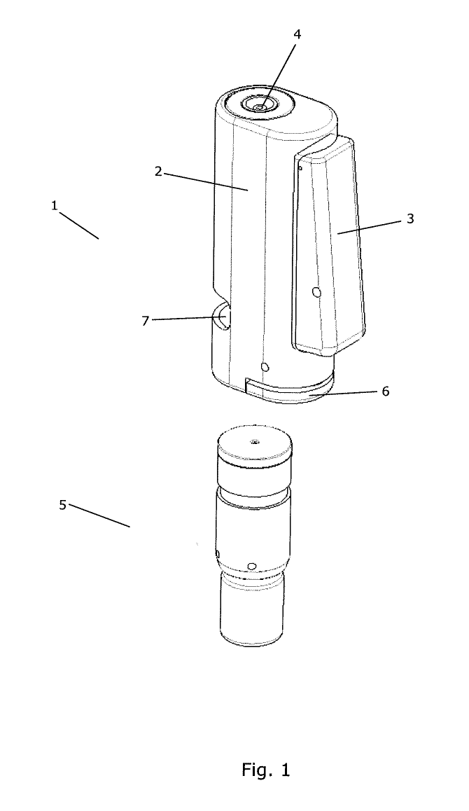

FIG. 1 is a perspective view of a drug delivery device 1 according to an embodiment of the invention. The drug delivery device 1 comprises a housing 2 being provided with a button 3 for operating a pump (not visible). The operation of the pump will be described in further detail below. The housing 2 is further provided with an outlet opening 4 adapted to receive a luer lock, thereby fitting tubing at the outlet opening 4. A liquid drug can be delivered from the drug delivery device 1 via the outlet opening 4. This will be described in further detail below.

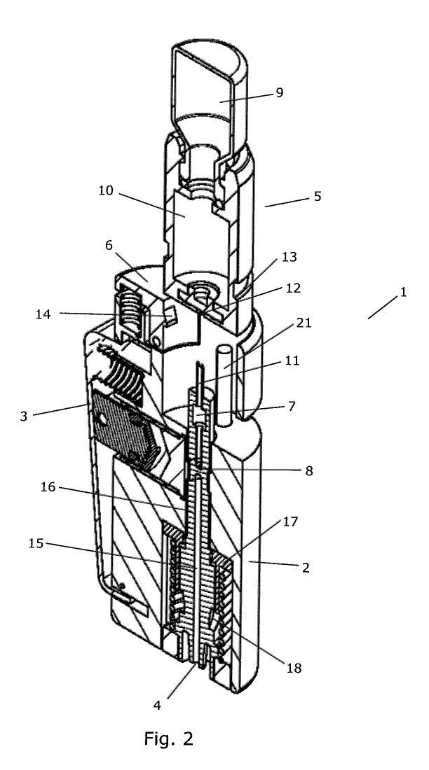

FIG. 1 further shows a reservoir 5 containing a liquid drug to be delivered. In FIG. 1 the reservoir 5 is shown detached from the drug delivery device 1. However, by pressing the drug delivery device 1 over the reservoir 5, the reservoir 5 will be inserted into the drug delivery device 1, thereby enabling the drug delivery device 1 to deliver the liquid drug contained in the reservoir 5. This operation is possible using only one hand if the reservoir 5 is positioned on a substantially flat surface, e.g. a table, or if the reservoir 5 is retained by other means. This will be described in further detail with reference to FIG. 2.

The drug delivery device 1 is further provided with a release mechanism 6 which, when pushed, will cause an inserted reservoir 5 to be released from the drug delivery device 1. This operation is also possible using only one hand. Accordingly, several reservoirs 5 may be consecutively inserted into and released from the drug delivery device 1, and the drug contents of several reservoirs 5 may thereby be delivered from the drug delivery device 1 without the user having to use more than one hand for operating the drug delivery device 1.

The drug delivery device 1 comprises an observation chamber 7 through which it is possible to monitor the liquid contents in the drug delivery device 1. Thereby it is possible for the user to see when a reservoir 5 is empty, ensure that aspiration is performed correctly, etc.

FIG. 2 is a cross sectional view along a longitudinal axis of the drug delivery device 1 of FIG. 1. In FIG. 2 the reservoir 5 is in the process of being inserted into the drug delivery device 1.

When a dose of liquid drug is to be delivered from the drug delivery device 1, the outlet opening 4 is initially connected to the vein of a person to receive the drug, e.g. via an infusion set comprising tubing and a butterfly needle (not shown). A valve element 8 will be in ‘aspiration mode’, i.e. operating the pump 3 will result in blood being drawn from the person into the drug delivery device 1. The pump 3 is operated until blood is visible in the observation chamber 7. Thereby it is ensured that the butterfly needle is actually positioned in a vein, and that the amount of air present in the tubing has been minimised.

The operation of the valve element 8 will be further described below.

The reservoir 5 comprises a vial 9 and an adaptor 10. The reservoir 5 contains the liquid drug and air in a ratio which allows for pumping of the liquid drug out of the reservoir 5 without venting the reservoir 5. When the reservoir 5 is inserted into the drug delivery device 1 a needle 11 penetrates a septum 12, thereby establishing a fluid connection to the interior of the adaptor 10, and thereby to the interior of the reservoir 5. During this process the reservoir 5 is retained in the drug delivery device 1 due to engagement between a groove 13 on the reservoir 5 and a tap 14 on the release mechanism 6.

During insertion of the reservoir 5 the valve element 8 is automatically moved into ‘infusion mode’. Accordingly, when the pump 3 is operated, the liquid drug is delivered from the reservoir 5 via the needle 11, a channel 15 and the outlet opening 4.

When the reservoir 5 is empty the release mechanism 6 is pushed, thereby causing the groove 13 and the tap 14 to disengage, and the reservoir 5 is thereby released from the drug delivery device 1, and a new reservoir 5 may be inserted. During the release of the reservoir 5 the valve element 8 is automatically moved into ‘aspiration mode’, and aspiration may therefore be performed before the next reservoir 5 is inserted. Thereby the amount of air present in the system is minimised prior to infusion of the next dose.

The valve element 8 is automatically moved between ‘aspiration mode’ and ‘infusion mode’ in the following manner. The valve element 8 is biased towards ‘aspiration mode’, i.e. when no reservoir 5 is inserted in the drug delivery device 1, the valve element 8 will be in ‘aspiration mode’. When a reservoir 5 is inserted it presses against push rod 21, thereby providing an axial movement of a guide 17. The guide 17 and a cylinder 16 are engaging via helical tracks 18, and the cylinder 16 is therefore forced to perform a rotational movement, thereby rotating the valve element 8. This rotation of the valve element 8 causes it to be moved into ‘infusion mode’. When the reservoir 5 is subsequently released from the drug delivery device 1, the cylinder 16 performs a reverse rotational movement, thereby causing the valve element 8 to be moved back into ‘aspiration mode’.

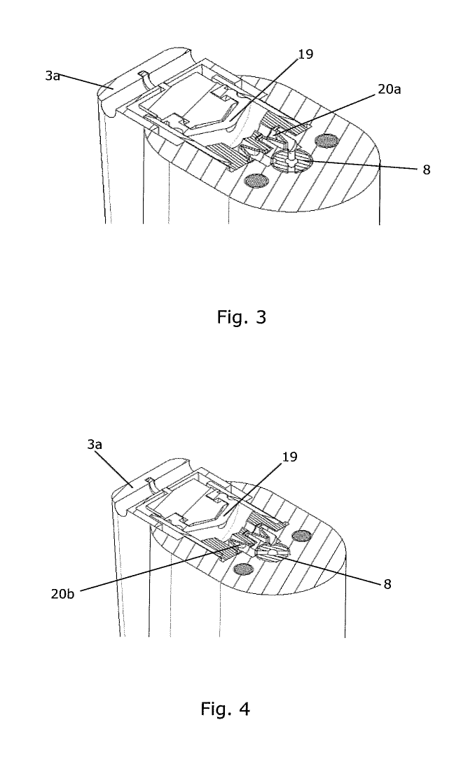

FIGS. 3 and 4 are cross sectional views along a transverse direction of the drug delivery device 1 of FIGS. 1 and 2.

FIG. 3 shows the valve element 8 in ‘infusion mode’. When the button 3a of the pump is pushed, piston 19 is moved forward, thereby causing liquid to enter the flow channel (15 in FIG. 2) via unidirectional valve 20a and the valve element 8. Thereby the liquid drug is delivered from the drug delivery device 1.

FIG. 4 shows the valve element 8 in ‘aspiration mode’. As compared to the situation in FIG. 3, the valve element 8 has been rotated in such a manner, that when the button 3a of the pump is pushed and the piston 19 moved forward, a pressure is build up. Accordingly, when the button 3a is released, liquid will be drawn from the flow channel (15 in FIG. 2) into the pump via the valve element 8 and unidirectional valve 20b.

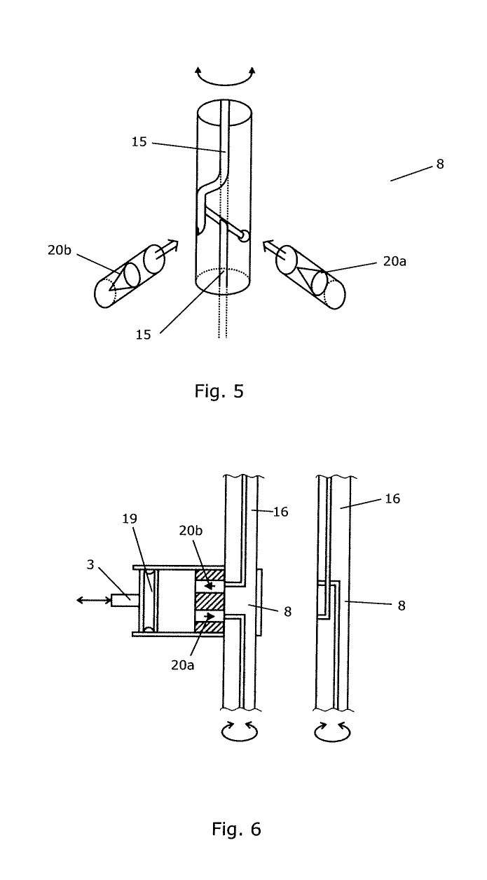

FIG. 5 is a perspective view of the valve element 8 shown in FIGS. 1-4. It is clear that rotating the valve element 8 causes a fluid connection to be established to one of the unidirectional valves 20a, 20b, thereby controlling the flow direction when the pump 3 is operated.

FIG. 6 shows an alternative valve arrangement in which rotation of the cylinder 16 causes connections to the unidirectional valves 20a, 20b to be switched, thereby controlling the flow direction. The left part of FIG. 6 shows the valve element 8 in ‘infusion mode’ and the right part of FIG. 6 shows the valve element 8 in ‘aspiration mode’.

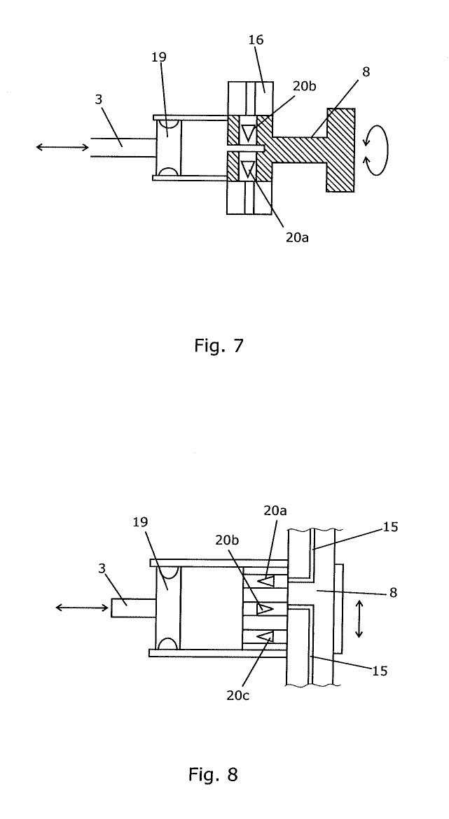

FIG. 7 shows another alternative valve arrangement. In the valve arrangement of FIG. 7 the valve element 8 is operated manually, and it is arranged perpendicularly to the cylinder 16. Rotating the valve element 8 through an angle of 180° causes the unidirectional valves 20a, 20b to be reversed, thereby reversing the flow direction during operation of the piston 19 of the pump.

FIG. 8 shows yet another alternative valve arrangement. The valve arrangement of FIG. 8 is operable by means of a linear movement of the valve element 8. In FIG. 8 the valve element 8 is positioned in such a manner that unidirectional valves 20a and 20b are connected to the flow channel 15. When the piston 19 of the pump is operated a fluid flow is thereby caused in a downwards direction in the Figure. Displacing the valve element 8 will establish fluid connections between the flow channel 15 and unidirectional valves 20b and 20c. When the piston 19 of the pump is operated in this case, a fluid flow is caused in an upwards direction in the Figure.

1. A drug delivery device comprising:
means for receiving a reservoir containing a drug to be delivered,
means for retaining a reservoir in the receiving means, said retaining means being adapted to be in a locking state in which a reservoir may be retained, and in an unlocking state in which the reservoir is released,
means for operating the retaining means to move between the locking state and the unlocking state, thereby causing a reservoir to be retained in or released from the receiving means,
an outlet opening adapted to deliver a drug from the device,
means for establishing a fluid connection between a reservoir retained in the receiving means and the outlet opening, thereby allowing a fluid flow between the reservoir and the outlet opening, and
pumping means for causing a flow of fluid in the established fluid connection.

2. A drug delivery device according to claim 1, wherein the pumping means is adapted to be in a first state in which fluid is allowed to flow from a reservoir retained in the receiving means towards the outlet opening, and in a second state in which fluid is allowed to flow from the outlet opening towards the reservoir, the pumping means being switchable between the first state and the second state.

3. A drug delivery device according to claim 2, wherein the pumping means is automatically switched between the first state and the second state in response to positioning or removing a reservoir in/from the receiving means.

4. A drug delivery device according to claim 2, wherein the pumping means is manually operable.

5. A drug delivery device according to claim 1, wherein the retaining means comprises releasable locking means.

6. A drug delivery device according to claim 1, wherein the retaining means comprises means for ejecting a reservoir being retained in the receiving means.

7. A drug delivery device according to claim 1, wherein the means for establishing a fluid connection comprises means for penetrating a reservoir being retained in the receiving means.

8. A drug delivery device according to claim 7, wherein the penetrating means comprises means for shielding the penetrating means when not penetrating a reservoir.

9. A drug delivery device according to claim 1, wherein the outlet opening is connectable to tubing for delivering the drug.

10. A drug delivery device according to claim 1, wherein the outlet opening is provided with needle means for delivering the drug.

11. A drug delivery device according to claim 1, wherein the drug delivery device is contained in a handle.
